(19) United States
(12) Patent Application Publication
(10) Pub. No.: US 2019/0133886 A1
(43) Pub. Date: May 9, 2019

(54) SYSTEM AND METHOD FOR DISPENSING MEDICATION

(71) Applicant: Kristine Brecht, Burien, WA (US)

(72) Inventor: Kristine Brecht, Burien, WA (US)

(21) Appl. No.: 16/180,336

(22) Filed: Nov. 5, 2018

Related U.S. Application Data
(60) Provisional application No. 62/583,674, filed on Nov. 9, 2017.

(51) Int. Cl. A61J 7/00; G16H 20/13; B65D 83/04; G05D 7/06

(52) U.S. Cl. CPC A61J 7/0076; G05D 7/0605; B65D 83/04; G16H 20/13

(57) ABSTRACT

A system for dispensing medication comprises: a plurality of compartments to contain medication for a patient, wherein the compartments are configured to dispense pre-determined doses of medication of the patient; biometric sensors configured to measure biological characteristics of the patient; a display configured to display medication-related information; and a processor. The processor is configured to: determine one or more dispense times, each dispense time corresponding to each dose of the pre-determined doses of medication; store of one or more parameters for dispensing the pre-determined doses of medication at the corresponding dispense times, wherein the parameters relate to the biological characteristics; receive the biological characteristics of the patient from the biometric sensors; compare the parameters with the biological characteristics received; determine whether to dispense at least one of the pre-determined doses of medication from the compartments; and dispense one or more of the pre-determined doses of medication from the compartments.

### CROSS-REFERENCE TO RELATED APPLICATION

This application claims priority to U.S. Provisional Application No. 62/583,674 filed Nov. 9, 2017.

### FIELD OF THE INVENTION

The field of disclosure is generally directed to a medication dispensing unit. More specifically, embodiments are provided in the present disclosure for a medication dispensing unit that is adapted to dispense medications at particular times according to a patient's recommended schedule and specific dosage amounts.

### BACKGROUND OF THE INVENTION

Whenever a physician prescribes medication, a correct dosage amount and medication schedule is provided for a patient to follow. In order for medication to be effective, a patient should adhere to the medication schedule and also be sure to take the correct amount of prescribed medication. Unfortunately, many patients do not follow the recommended schedule provided by their physician, and often end up taking too much or too little of their medications. This results in the medication not being effective because it is not used properly, and it can also mean that there may be serious, negative side effects as a result of a patient's improper administration of medicine.

Many patients do not comply with proper usage of prescription medications for a variety of reasons. Patients often improperly take their medication simply because they forget when to take their medication. It is also possible that patients do not understand their schedule. In addition, many patients are prescribed multiple medications for various conditions. This means that patients may regularly have complicated medication schedule (e.g. taking a dose of medication every four hours, increasing or decreasing a number of pills over a period of time, only taking a medication if needed etc.), and due to the complexity and variability of the timing of the medications, it becomes a common issue that patients may not take their medication at the correct times or in the correct dosage amounts. Further, sometimes a patient may improperly ingest or consume their prescription medication as a consequence of the patient not realizing a risk of a harmful interaction with another medication being taken at the same time, or by a patient continuing to take a medication without realizing that he or she is experiencing a harmful side effect.

There have been some attempts at providing medication dispensation units that assist a patient in taking their medication. However, they have many disadvantages and short comings. For example, the conventional pill boxes often do not provide reminders to a patient when to take medication or how much. Further, they do not take into consideration that there are certain types of medications that should not be taken (e.g. blood pressure medication or sedation related medication) unless a patient is in the proper condition and/or state of health for taking these types of medication.

In addition, it has recently become a major epidemic in the United States and elsewhere around the world for certain pain-relieving medications, such as oxycodone (or other opioids), to be abused by patients taking too much at one time thinking that this increased amount will better assist them with managing any pain they are experiencing. The same potential for abuse is frequently occurring with sedation medication. Alternatively, it is also a serious problem that friends or family members who have access to such medications may end up taking these medications even though they are not authorized to and are not the intended patient. The medication pill boxes and dispensing containers oftentimes lack the proper safety features to prevent such medications from being abused or administered, whether intentionally or not, to the wrong person.

Therefore, there currently exists a critical need in the industry for a medication dispenser that is better able to assist patients in the proper taking of their medication in the right amounts and at the correct time, which also utilizes one or more mechanisms for verifying an identify of the patient, while also taking into consideration a way to monitor the state of the health of the patient in order to ensure that the medication will be effective before taking the medicine unnecessarily.

### SUMMARY OF THE INVENTION

In one aspect, embodiments are provided in the present description for a system for dispensing medication. In some exemplary embodiments, the system may include a plurality of compartments to contain medication for a patient. The medication may be in the form of pills, tablets, or combination thereof (including any form known in the art such as gels, capsules, etc.).

Further, in some embodiments, the plurality of compartments may be configured to dispense one or more pre-determined doses of medication of the patient. Alternatively, in some embodiments, each of the plurality of compartments may contain one kind of medicine and be configured to dispense a pre-determined sub-dose of medication of the patient, where the sub-doses from the plurality of compartments are combined to make a pre-determined dose of medication.

The system may further include one or more biometric sensors, such as, but not limited to a fingerprint scanning device that may be used to verify the identity of a user trying to access the medications before dispensing or granting access to the medications. In some embodiments, the one or more biometric sensors may include a pulse oximeter and a blood pressure monitoring device. The biometric sensors may be physically integrated into the system as a medication dispensing unit, or they may be connected to the medication dispensing unit configured to communicate measurements with the medication dispensing unit.

The system may further include a display that displays information related to medication, such as visual instructions and/or visual alerts that act as reminders to a patient regarding the taking of the medication and/or provide additional helpful information.

Further, the system may include one or more processors that are configured to determine when to dispense a patient's medication according to the patient's medication schedule, including to determine what time to dispense a dose of medication and whether to dispense at certain intervals. The one or more processors may also be configured to store parameters for dispensing the pre-determined doses of medication at the corresponding dispense times, where the parameters are related to the biological characteristics; receive the biological characteristics of the patient from the biometric sensors; compare the parameters with the biological characteristics; and determine whether to dispense a pre-determined dose of medication from the compartments. The parameters may include the patient's pulse, blood pressure level, and/or oxygen saturation level.

In some embodiments, the one or more processors may also be configured to send notifications to the patient about his or her dispensing schedule. The notification may be timely given to the patient using the display, or it may be sent to the patient in various ways including, but not limited to, text messages, audible alarm, visual alarm, and email messages.

Further, in some embodiments, the system may include a user interface configured to receive one or more user inputs. The one or more user inputs may include a patient identification. A user may use the user interface to interactively control the system by communicating with the one or more processes, or to input various information, including but not limited to, a patient's identification, medical record, crucial health risk specific to the patient, and/or the information regarding the patient's healthcare provider.

In another aspect, embodiments are provided in the present description for an exemplary method for storing and dispensing medication from a medication dispensing unit. In one embodiment, the non-limiting, exemplary process may include providing a medication schedule for a patient and configuring the medication dispensing unit to dispense the medication to the patient according to the medication schedule of the patient. Further, the process may include verifying an identify of the patient.

Additionally, the process may further include, in some embodiments, configuring the medication dispensing unit to receive measurements of biological characteristics from a patient prior to dispensing particular medications for the patient. As used herein, “biological characteristics” may refer to a patient's fingerprint and/or any other vital signs of the patient. For example, a medication dispensing unit may be configured to receive measurements regarding a patient's vital signs such as blood pressure level, oxygen saturation level, and pulse. Responsive to the received measurements of a patient's blood pressure, oxygen saturation level, and pulse, the process may include determining whether a threshold trigger level has been reached for the received vital signs of the patient. If the threshold trigger level is reached, then the medication is not dispensed to the patient and the data regarding the received measurements may be provided to a healthcare provider. As used herein, a “healthcare provider” may refer to a physician, caregiver, a pharmacist, or any other party relevant to the health of a patient. Alternatively, or additionally, if the threshold trigger level for any of the vital signs is not reached, then the medication is dispensed to the patient according to the medication schedule and after having verified the identity of the patient.

The foregoing summary is illustrative only and not intended to be in any way limiting. Other aspects and advantages of the invention will be apparent from the following description and the appended claims.

### BRIEF DESCRIPTION OF DRAWINGS

The present invention will be described by way of exemplary embodiments, but not limitations, illustrated in the accompanying drawings in which like references denote similar elements, and in which:

FIG. 1 is a block diagram illustrating exemplary components of a system for a medication dispensing unit according to embodiments of the present disclosure.

FIG. 2 is a pictorial illustration of an exemplary medication dispensing unit according to embodiments of the present disclosure.

FIG. 3 is a pictorial illustration of another exemplary medication dispensing unit according to embodiments of the present disclosure.

FIG. 4 is a flowchart for an exemplary process of dispensing medication after receiving vital signs measurements according to embodiments of the present disclosure.

### DETAILED DESCRIPTION OF THE INVENTION

In the Summary above and in this Detailed Description, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range including that number (which may be a range having an upper limit or no upper limit, depending on the variable being defined). For example, “at least 1” means 1 or more than 1. The term “at most” followed by a number is used herein to denote the end of a range, including that number (which may be a range having 1 or 0 as its lower limit, or a range having no lower limit, depending upon the variable being defined).

“Exemplary” is used herein to mean “serving as an example, instance, or illustration.” Any aspect described in this document as “exemplary” is not necessarily to be construed as preferred or advantageous over other aspects.

Throughout the drawings, like reference characters are used to designate like elements. As used herein, the term “coupled” or “coupling” may indicate a connection. The connection may be a direct or an indirect connection between one or more items. Further, the term “set” as used herein may denote one or more of any item, so a “set of items,” may indicate the presence of only one item, or may indicate more items. Thus, the term “set” may be equivalent to “one or more” as used herein.

In the following detailed description, numerous specific details are set forth in order to provide a more thorough understanding of the one or more embodiments described herein. However, it will be apparent to one of ordinary skill in the art that the invention may be practiced without these specific details. In other instances, well-known features have not been described in detail to avoid unnecessarily complicating the description.

The present disclosure is generally drawn to a system and method, according to one or more exemplary embodiments, for a medication dispensing unit that is adapted to assist a patient in remembering when to take their medication in an organized fashion. Further, useful features of the one or more embodiments includes that the medication dispensing unit may require, at least in some non-limiting embodiments, information regarding important vital signs of the patient prior to the administering of the medicine from the medication dispensing unit. This may be helpful to prevent a patient from taking medication that, while normally is beneficial, may have adverse consequences based on the current state of the patient's health. Further details about the medication dispensing unit according to one or more embodiments shown in the Figures are provided below.

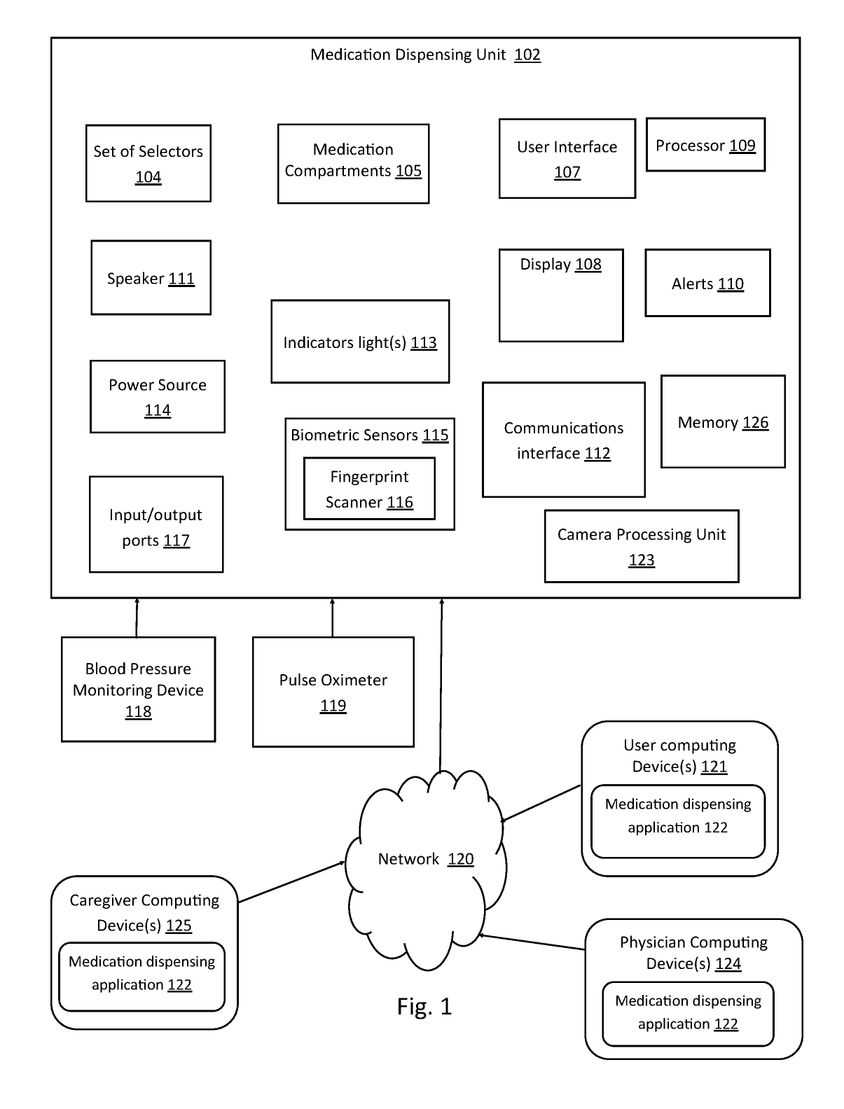

Turning to FIG. 1, FIG. 1 is a block diagram of a medical dispensing unit and some examples of components that may be included in a system of medication dispensing unit, such as medication dispensing unit 102. In one or more embodiments, medication dispensing unit 102 is an apparatus configured to hold and dispense one or more doses of medication to a patient in a pre-determined order and according to a set schedule. The timing and the order of the medications may be obtained from a schedule that is prepared by a patient's physician, nurse, or other healthcare provider. As used herein, the term “medication” or “medications” includes medications in the form of pills, tablets, capsules, gel caps, and any other dosage unit known in the art for administering pharmaceutical agents. In some embodiments, the medication may be oral medication that is in a generally solid dosage form (or may be enclosed in a solid casing as in the case of liquid gel caps). Further, the term “medication” or “medications,” as used herein, may include vitamins and/or any type of dietary or nutritional supplements. Further, the term “dose” and/or “doses,” as used herein, may refer to a quantity of a medication taken or recommended to be taken at a particular time. A dose may further be comprised of “sub-doses,” where a sub-dose refers to a quantity of a specific kind of medicine in a dose of medication. In other words, a combination of sub-doses of different kinds of medicine makes a dose of medication at a particular time.

Further, the term “patient” as used herein may be interchangeable with a user. More particularly, a patient is an intended user of medication to be stored in medication dispensing unit 102. In one or more embodiments, medication dispensing unit 102 may be assigned to correlate to a single user or patient and cannot be used by more than one person at one time for purposes of storing medications and dispensing the medications. However, this does not preclude that a non-patient user, such as a patient's caregiver, nurse, or physician, or other relevant party may still have access to the contents of medication dispensing unit 102 for purposes of assisting and/or monitoring the patient while using medication dispensing unit 102.

Thus, medication dispensing unit 102 is a device capable of holding and storing medication, and is further capable of automatic dispensation of the medication at pre-set times according to a preferred medication regimen and schedule. Further, medication dispensing unit 102, in one or more embodiments, is capable of receiving, processing, and/or providing information via the one or more components included in FIG. 1 and further described below.

In one or more embodiments, medication dispensing unit 102 has a housing, that encloses one or more components included within medication dispensing unit 102. Housing may include a single casing and/or interconnectable casings that provide structural support for medication dispensing unit 102. Housing may be made of a plastic material in one or more preferred embodiments. However, those of ordinary skill in the art will appreciate that housing and medication dispensing unit 102, in general, may also include or be made of other varying types of materials, including metal, glass, and/or another material other than or in addition to plastic.

Medication dispensing unit 102 may further include at least one set of selectors 104 which may be any type of selectors known in the art, including push buttons, touchscreen buttons, toggle slides, knobs, or any other suitable selectors that may be used to interface with one or more components of medication dispensing unit 102. Any number of selectors may be included with medication dispensing unit 102 that correspond to various useful functions, as further discussed below.

Further, medication dispensing unit 102 may further include a user interface, such as user interface 107 and a display, such as display 108. User interface 107 may be any type of user interface known in the art, including touchscreens, keyboards having alphanumeric characters, or any other user interface known in the art. Further, display 108 may also be in some cases a monitor or screen, including a touch screen, that may function to display one or more instructions or other helpful information to a patient on the medication dispensing unit 102. Display(s) 108 may be any number of touchscreens, monitors, or other display device capable of displaying text, images, symbols, and/or any other type of desired information. Display(s) 108 may be capable of displaying multimedia data from medication dispensing unit 102 or other devices (e.g. blood pressure monitoring device 118, pulse oximeter 119, or computing devices 121, 124, and 125).

It is an object of the one or more embodiments provided in the present description that medication dispensing unit 102 is suitable to handle medication dispensing schedules that range from relatively simple, straightforward types to more complex types. For non-limiting, exemplary purposes only, some patients are asked to take anywhere between three to seven different medications at a time in order to treat one or more conditions or disorders. There may be a medication or set of medications that a patient is prescribed to take every morning or every evening. There may also be medications that should be taken at least twice a day, once in the morning and once in the evening. There may also be medications that are recommended to be taken in intervals, such as every two hours, every four hours, every eight hours, etc. Therefore, it is not hard to see why many patients have difficulty in keeping their medication regiment. Further, it is not hard to see how many patients may easily forget whether they have actually taken a medication on time or not, particularly if the patient has a more complex medication regimen, each time having several different kinds of medicine. Existing medication dispensing units are usually only able to handle very simple, straightforward medication dispensing routines or schedules, and are not designed to provide medications that have to be dispensed at particular intervals (e.g. every two hours, every four hours, or every eight hours).

Medication dispensing unit 102 may include a plurality of medication compartments 105. In some non-limiting exemplary embodiments, medication compartments 105 are individualized containers or compartments that allow the patient to compartmentalize his or her medications in an ordered fashion and per the desired dose per compartment. Each of the desired doses of medication within a compartment may be comprised of sub-doses of specific kinds of medicine.

Alternatively, in some embodiments, medication compartments 105 are individualized containers or compartments where compartment contains a specific kind of medicine to be later combined into a dose of medication suitable for a specific medication time per medication schedule. Processor 109 may be configured to open each of medication compartments 105, extract a sub-dose of medication pre-determined per medication schedule, and combine the extracted sub-doses to dispense a dose of medication to the patient according to medication schedule at the pre-determined dispense time.

Regardless of specific embodiment of medication compartments 105, medication dispensing unit 102 may be configured to have a variety of number of compartments and a variety of sizes of compartments and accommodate a variety of medication regimens. Medication dispensing unit 102 may be capable of storing and dispensing a one day supply of medication, or a one week supply, or a one month (30 day) supply, or longer such as a 60 day supply dispensing schedule. Those of ordinary skill in the art will appreciate that or any amount of time greater or less than these above-listed scheduled duration of time may also be feasible.

It is noted that medication dispensing unit 102 may be configured to determine medication at a certain or specific time, and is also able to calculate dispensing based on intervals. Thus, if a medication schedule indicates that a medication should be taken every four hours, for example, the medication dispensing unit 102 is able to calculate the specific time to dispense a medication from a starting time.

Accordingly, medication dispensing unit 102 may include a processor such as processor 109. Processor 109 of medication dispensing unit 102 may include a number of electronic and electromechanical components configured to determine the order and the timing of dispensation of a set of medications contained within medication dispensing unit 102. For example, medication dispensing unit 102 may be programmable to receive one or more instructions regarding a schedule for dispensing medications, including an order and a timing for dispensing medications from medication compartments 105. Further details regarding processor 109 are included below.

Further, medication dispensing unit 102 may be capable of providing alerts, such as alerts 110, that alert a patient as to the time to take a medication. Thus, alerts 110 may serve as helpful reminders to the patient prior to taking medication at a certain time. Alerts 110 may be any type of alert, including audio alerts that either provide sounds, such as beeping noises or alarms of varying levels. Alternatively, or additionally, alerts 110 may include voice recordings that verbalize any number of words that constructively and effectively remind a patient about the timing. Alerts 110 may further include information for the name of the medication that is being dispensed at a particular time. Alerts 110 may also be visual alerts that are displayable on display 108. Basically, alert 110 is any type of alert that is capable of drawing a patient's attention that the medication is about to be dispensed. Accordingly, when alert 110 is a visual alert, there may be any type of text, images, and/or symbols provided on display 110 to attract a patient's attention in addition to audible alerts that are emitted from one or more speakers, such as speaker 111, included with medication dispensing unit 102.

Speaker 111 may be any type of suitable speaker known in the art. Speaker 111 may usefully output any sound, including audible alerts, such as alerts 110, or other helpful instructions for the patient. Further, n some embodiments, medication dispensing unit 102 may be capable of issuing voice instructions as recorded by either the patient, a caregiver, a physician, a pharmacist, or other relevant party that provides useful instructions to the patient regarding the medications and medication regimen associated with the patient. For examplary purposes only, the voice instructions that may be pre-recorded and stored in memory 126 of medication dispensing unit 102 may provide reminders to take a particular type of medication with food so as to avoid any trouble when ingesting the medication.

In one or more embodiments, alerts 110 may be provided at varying time periods prior to dispensing the medications. For example, alerts 110 may be provided fifteen to ten minutes prior to the scheduled time for dispensation from medication dispensing unit 102 is to occur, although any other time period for providing an alert may also be used. In one or more embodiments, the patient or another party is able to program when the alerts 110 are to be triggered in medication dispensing unit 102.

Further, speaker 111 may also output various verbal messages to the patient such as, but not limited to, access to medications granted, access to medications denied, new message received, time to take medication, etc. In addition to the above, a button or other type of selector from the set of selectors 104 included on an outer surface of medication dispensing unit 102 may be provided that allows for a user to silence the one or more alerts 110 emitted from speaker 111 or displayed on display 108 of medication dispensing unit 102.

Medication dispensing unit 102 may include one or more indicator lights, such as dispensing indicators lights 113. Dispensing indicator light 113 may be any type of light included with medical dispensing unit 102 that is adapted to light up once the medication is set to be released from medication compartments 105. Alternatively, or in addition, one or more light emitting diode (LED) lights may be located on each medication container that lights up when the medication is set to be released from medication compartments 105.

In one or more embodiments, power source 114 provides power to medication dispensing unit 102. Power source 114 may provide power for operating medication dispensing unit 102. In some embodiments, power source 114 may include an electrical wire configured to be plugged into an electrical outlet separately located in the location where a patient keeps medication dispensing unit 102. Alternatively, or additionally, medication dispensing unit 102 may also include a battery housing adapted to house one or more batteries, which may be either rechargeable or non-rechargeable.

Additionally, medication dispensing unit 102 is configured to prevent a patient from accessing too much of a medication. It is to be appreciated that prescription medication abuse and misuse is a very public health issue in the United States and elsewhere in the world. It is an object of one or more embodiments of medication dispensing unit 102 to reduce the potential for prescription medication abuse. As used herein, “medication abuse”, may refer to the improper consumption of prescription medication including taking a larger dose than a patient is prescribed. Additionally, “medication abuse” may refer to taking medication that one is not supposed, which may happen when a patient or another user attempts to share (including by selling) their medication with other users who are not authorized to have such prescription medication. Some examples of prescription medication that are commonly abused are opioids, sedatives, tranquilizers, and stimulants. Various types of antidepressants may be included within these categories of medications as well and are also known for being improperly administered and misused. Such medications (which are usually prescription medications) tend to be very commonly abused and misused.

When physicians prescribe such medications, they do so knowing that these medications may provide beneficial relief and may assist in treating one or more conditions or disorders that a patient is experiencing. However, if a patient takes too much of any of these medications and in an incorrect manner, many serious, harmful side effect may occur. Furthermore, it is known that the above listed types of commonly abused medications, including, but not limited to, opioids, sedatives, tranquilizers, and stimulants, may be addictive in nature and habit forming. Thus, it is a serious concern that when a patient is provided with, for example, a pill container having several units of these medications that the patient may, intentionally, take more than he or she is supposed to according to the physician's prescriptions.

In one or more embodiments, medication compartments 105 may be designed to be opened only with authorization from a physician or other caregiver. Alternatively, or additionally, medication compartments 105 may be designed to include lockable lids that may be released only with authorization and/or if it is determined by a processor located within medication dispensing unit 102 (e.g. processor 109) that the patient is able to receive this particular dosage of medicine at a certain time.

Further, medication dispensing unit 102 may be configured to include one or more biometric sensors, such as biometric sensors 115. Biometric sensor(s) 115 may include any type of biometric device that identifies a biometric feature of a user. In one or more embodiments, biometric sensors 115 may be a biometric device particularly suited for identifying a fingerprint of a patient, such as a fingerprint scanner 116 that detects a fingerprint of the patient. In one or more embodiments, medication dispensing unit 102 may have a database of fingerprints of specific patients who will be taking the medication. The database may be stored in memory, such as memory 126, connected to processor 109 and placed within medication dispensing unit 102. Alternatively, medication dispensing unit 102 may require and/or allow a user to pre-register the fingerprints of a patient who will be taking the medication.

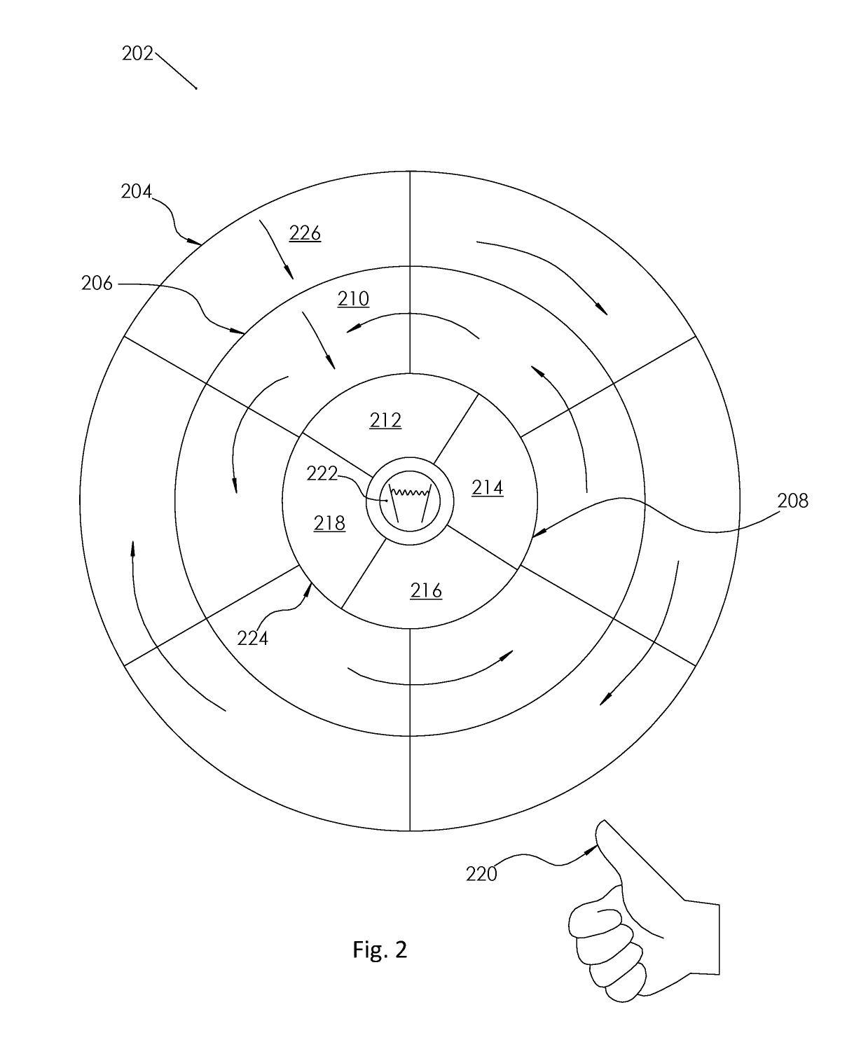

Accordingly, medication dispensing unit 102 may require that a patient initially associate one or more fingerprints of the patient when first setting up medication dispensing unit 102 to be associated with the patient. This may be useful for preventing prescription medication abuse and misuse by any unauthorized users who attempt to access any of the medication contained within medication dispensing unit 102. For example, a user may press a designated finger (e.g. finger 220 in FIG. 2) or one or more fingers on a corresponding location of a fingerprint scanner in order to trigger any sort of response from the medication container 102. For example, the authorized user may be required to provide his or her fingerprint in order for a lid or medication dispensing door (in one or more embodiments) to be unlocked and for the user to be provided with access to the medication located inside.

Other biometric devices may also be included with medication dispensing unit 102 that identify other biometric features of the patient, including, but not limited to, detecting an eye pattern or a vein pattern of the patient, or any other type of biometric sensing device known in the art. Alternatively or additionally, medication dispensing unit 102 may include one or more input/output ports, such as input/output ports 117 for various devices such as biometric devices to connect to medication dispensing unit 102.

In addition to the above, in some embodiments, medication dispensing unit 102 may include a camera processing unit 123. Camera processing unit 123 may include any suitable camera having image recording, including video recording capabilities. In one or more embodiments, multimedia data obtained by camera processing unit 123 may be transmitted and provided as a live feed for a physician or other health care provider associated with physician computing device 124 (or alternatively a pharmacist or another type of healthcare provider) and/or a caregiver (e.g. family member or friend) associated with caregiver computing device 125 to view on one or more display user interfaces. In some cases, it may be more reassuring to the healthcare provider such as caregiver and/or physician to be able to monitor that the medication was properly dispensed and actually taken by the patient by requiring the patient to record him or herself during the consuming and taking of the medication in front of the camera processing unit 123. Alternatively, there are many other possible applications for camera processing unit 123. For example, a physician or caregiver may be able to record him or herself recording instructions for the patient and the patient may be able to select and view such recordings, in one or more embodiments.

A useful object of the one or more embodiments of medication container 102 is to be able to verify that the patient should take the medications without enhancing any adverse health conditions. There have been instances where a patient who is prescribed blood pressure medication has suffered from respiratory failure after taking his or her medication, if, for example, the patient's oxygen saturation level is less than 88% (in one non-limiting example). There are many other instances in which if a patient has low blood pressure or high blood pressure or a low or high pulse rate for there to be adverse reactions to taking different types of medications.

Accordingly, in one embodiment, prior to dispensing the medication from medication dispensing container 102, a patient may be required to take and share his or her vital signs. This may be used to monitor the state of the health of the patient and to prevent adverse consequences if a patient takes medication that may contribute to respiratory failure, heart failure, or another disorder.

In particular, a patient may be required to enter or otherwise provide his or her blood pressure, pulse, and oxygen saturation level. Thus, medication dispensing unit 102 may be connectable to one or more medical devices such as blood pressure monitoring device 118 and pulse oximeter 119. Alternatively, blood pressure monitoring device 118 and/or pulse oximeter 119 may be installed within medication dispensing unit 102. Blood pressure monitoring device 118 may also be referred to as a sphygmomanometer by those skilled in the art, and may be used to measure blood pressure of the patient. Blood pressure monitoring device 118 may be, in a non-limiting example, an inflatable cuff having a measuring unit and a mechanism for inflation which may be a manually operated bulb and valve or a pump operated electrically.

Pulse oximeter 119 is another medical device that may be connected to medication dispensing unit 102. Pulse oximeter 119 may measure the oxygen saturation level for a patient. Additionally, pulse oximeter 119 may measure the pulse or the heart rate of a patient. A person's pulse is a measurement of the heart rate of the individual. Any suitable pulse oximeter as available in the art may be used for pulse oximeter 119, including the finger pulse oximeters as known in the art.

In one embodiment, blood pressure monitoring device 118 and pulse oximeter 119 may be connectable to medication dispensing unit 102 and the readings or measurements obtained from these devices provided directly to a processing unit of medication dispensing unit 102. The processing unit of medication dispensing unit 102 may compare the readings and measurements obtained from blood pressure monitoring device 118 and pulse oximeter 119 in order to determine whether a threshold level for oxygen saturation level, blood pressure, and/or pulse rate has been exceeded.

Alternatively, in other embodiments, a patient may be required to enter in the most recent reading or measurement for his or her oxygen saturation level, blood pressure, and/or pulse. The patient (i.e. or another person such as a caregiver) may enter in the relevant numerical values obtained from blood pressure monitoring device 118 and pulse oximeter 119 using user interface 107 (e.g. touchscreen, keyboard) and responding to prompts for such information on the display 108 of medication dispensing unit 102.

In one embodiment, a threshold level for each vital sign to be measured against or compared to may be stored and used in an analysis by the processing unit 109 of medication dispensing unit 102 of whether the vital sign measurements provided from the patient exceed the threshold level or not. These threshold levels may be pre-loaded for an average patient. Alternatively, the threshold levels may be pre-loaded for an average patient of specific age, sex, weight, and other preconditions. Still alternatively, a user may be required to enter the threshold levels that are suitable to a specific patient who will be taking the medication.

If the threshold trigger level is reached, then a physician may be concerned for a patient to take certain medications. As described above, consuming certain medications may have adverse health effects on a patient if a patient already has a very low or very high blood pressure, oxygen saturation level, and/or pulse. Such medications include pain medications (e.g. opiates) which can depress breathing, which is why it is important to monitor one's oxygen levels. If a patient's oxygen level is low, taking such medications could compromise the patient's respiratory functions and may lead to respiratory failure, and possibly death in extreme circumstances. Further, it may be useful to take the pulse in order to know if a patient's heart rate is too low or too high, prior to taking certain medications that may aggravate their condition.

Thus, in some embodiments, a minimum and/or maximum threshold trigger level may be provided to medication dispensing unit 102 for such vital sign measurements to be compared against. For example, if an oxygen saturation level is less than 88%, the medication dispensing unit 102 may be configured so as not to dispense all or some of the stored medications in the medication dispensing unit 102.

Further, in some embodiments, the pulse may be set to be within a range of 50-140 beats per minute (bpm). Thus, the minimum pulse rate may be 50 bpm and the maximum pulse rate may be set to 140 bpm. If either number is reached or exceeded, then medication dispensing unit 102 may be configured so as not to dispense all or some of the stored medications in the medication dispensing unit 102.

Further, the blood pressure may be set to a value of 90/60, and if the blood pressure is less than this value, the medication dispensing unit 102 may be configured so as not to dispense all or some of the stored medications in medication dispensing unit 102. Those of ordinary skill in the art will appreciate that other values and limits may also be utilized, and that the values provided above are only meant to be exemplary and non-restrictive in nature.

Oftentimes, physicians and other caregivers are not able to properly monitor the taking of the medication in current dispensing containers. However, in one or more embodiments, physicians and other caregivers may be provided with an actual accounting of a patient's medication consumption and administration from medication dispensing unit 102. Medication dispensing unit 102 may be configured to transmit and communicate information about the timing and type of medication has been dispensed to the patient.

A processor, such as processor 109, may execute instructions to store such information in memory 126 of medication dispensing unit 102. Further, the information related to a timing of distribution and amount of medication taken by a patient may be communicated to one or more relevant parties to the patient, such as, without limitation, to a physician who prescribed the medications or to a caregiver of a patient. The term “caregiver” as used herein may refer to a family member, including spouse or partner, friend, or any other person of relevance to the patient, including nurses, home caregivers, and/or physical therapists.

Accordingly, in one or more embodiments, information relating to a history of the timing and amount of medication dispensed to the patient associated with medication dispensing unit 102 may be shared and transmitted to physician computing device(s) 124 and/or any other type of healthcare provider deemed necessary. Further, the information related to a history of the timing and amount of medication dispensed to the patient may also be transmitted to caregiver computing device(s) 125. Such information may be transmitted over network 120 using one or more components of communication interface 112 (e.g. a transceiver-like device). Those of ordinary skill in the art will appreciate that physician computing device (s) and/or caregiver computing device(s) may be any type of computing device known in the art, including, one or more desktop computers, laptops, notebooks, tablets, smart phones, wearable watches or the like, or any other suitable computing device.

Further, it is an object of one or more embodiments of the medication dispensing unit 102 for the patient to be provided with educational material and information about the type of medication he or she is asked to consume. Oftentimes, a physician, nurse, or other health care provider has a limited amount of time to talk with a patient and has to cover a great deal of subject matter. Usually, a patient is told what type of medications he or she is recommended or prescribed to take and may also be provided with fact sheets or informational pamphlets about the medication that the patient is prescribed. Unfortunately, most patients tend not to remember to review such information included in these provided sheet and/or eventually lose or misplace these items.

To provide additional education for the patient about the medications that he or she is asked to take, in one or more embodiments, a local application, such as medical dispensing application 122 may be downloaded onto any type of computing device, such as, but not limited to user computing device(s) 121, physician computing device(s) 124, and/or caregiver computing device(s) 125. The term “computing device(s)” as used herein may be any type of computing devices including, one or more computers, laptops, notebooks, tablets, smart phones, wearable watches or the like, or any other suitable computing device.

Medical dispensing application 122 may be a modular software application that is downloadable and installed onto any type of computing device, including user computing device 121, physician computing device 124, and/or caregiver computing device 125. Medical dispensing application 122 may be a type of local or native application that is stored in the memory of these computing devices. Alternatively, medical dispensing application 122 may be accessible over a cloud computing environment or network.

Medical dispensing application 122 may be in electrical and/or signal communication with medication dispensing unit 102 so that medical dispensing application 122 may send and receive relevant information to and from medication dispensing unit 102. For example, in one embodiment, it is possible that medical dispensing application 122 is configured to receive from medication dispensing unit 102 a list of medications that are contained within medication dispensing unit 102 (e.g. contained in medication compartments 105). Accordingly, in one embodiment, a patient receives educational information about the type of medicines that are included within medication dispensing unit 102 on a display of user computing device(s) 121. In other words, on a smartphone or tablet (for example), in one or more embodiments, when it is time for a patient to take a particular medication or grouping of medications, the medication dispensing application 122 may also display simultaneously information related to the medications on the display (e.g. screen or monitor) of user computing device(s) 121 and/or on display 108 of medication dispensing unit 102.

To provide these medications into user computing device(s), it is possible that the patient enters the name of the medications directly in medication dispensing application 102 and/or in user computing device(s) 121 using a user interface and display for these respective devices. Thus, medication dispensing application 102 may have a pre-loaded set of information about these medications or may query a server or other device for further data about the medications. Educational and helpful information that may be shared with the patient via medication dispensing application 122 may include information regarding the various names of conditions and disorders that the medications may be used to treat including symptoms of one or more conditions and disorders, the intended benefits of the medications, as well as any side effects. It is an object of the one or more embodiments for medication dispensing unit 102 that the patient is provided with a very functional, well-integrated dispensing unit 102 that integrates well with the user's computing devices 121, physician computing devices 124, and/or caregiver computing devices 125.

Further, in one or more embodiments, medication dispensing application 122 may provide reminder alerts that are emitted from a speaker or displayed on a display of user's computing device(s) 121 and/or caregiver computing device(s) 125. Thus, a caregiver may also be notified, in addition to the user, and provided with reminders that a medication is to be dispensed at a particular time. This may be particularly helpful for patients who are elderly and perhaps forgetful, such that the caregiver is able to contact them and remind them that their medications are being dispensed from the medication dispensing unit 102 and that it may be time to take them. Further, the user may directly receive additional reminders via the medication dispensing application 122 located on his or her phone, tablet, or other user computing device 121.

To continue with additional details for some of the components provided above, processor 109 may be any type of processor known in the art. Processor 109 may include, but is not limited to, a single or multi-core central processing unit (CPU), a special purpose processor, a conventional processor, a Graphics Processing Unit (GPU), a digital signal processor (DSP), a plurality of microprocessors, one or more microprocessors in association with a DSP core, a controller, a microcontroller, one or more Application Specific Integrated Circuits (ASICs), one or more Field Programmable Gate Array (FPGA) circuits, an other type of integrated circuit (IC), and/or a system-on-a-chip (SOC).

Processor 109 may be capable of processing and/or routing information to various components of medication dispensing unit 102 and to other computing devices, such as, but not limited to, user computing devices 121, physician computing device(s) 124, and also caregiver computing devices 125. Further, in some embodiments, processor 109 may also be capable of processing and/or routing information or receiving information (e.g. data regarding biological characteristics measurements) from blood pressure monitoring device 118 and pulse oximeter 119.

Medication dispensing unit 102 may further include communications interface 112 which may include a transceiver-like component (e.g. a transceiver, a separate receiver, and transmitter, or the like) that enables medication dispensing unit 102 to communicate with other devices, such as via a wired connection, a wireless connection, or a combination of wired and wireless connections. Communication interface 112 may permit medication dispensing unit 102 to receive information from another device and/or provide information to another device. For example, communication interface 112 may be used to send and/or receive information, including but not limited to, text, pictures, voice data, and voice recordings. In some implementations, communication interface 112 may include one or more of the following: an Ethernet interface, an optical interface, a coaxial interface, an infrared interface, a radio frequency (RF) interface, a universal serial bus (USB) interface, a Wi-Fi interface, a cellular network interface, or the like.

Medication dispensing unit 102 may perform one or more processes described herein. Medication dispensing unit may perform these process in response to processor 109 executing software instructions stored by a computer-readable medium, such as memory 126 and a storage component. A computer-readable medium is defined herein as a non-transitory memory device. A memory device includes memory space within a single physical storage device or memory space spread across multiple physical storage devices.

Software instructions may be read into memory 126 and an associated storage component from another computer-readable medium or from another device via communication interface 112. When executed, software instructions stored in memory 126 and/or storage component may cause processor 109 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

Network 120 may include one or more wired and/or wireless networks as known to those skilled in the art. For a non-limiting example, network 120 may include a cellular network, a public land mobile network (PLMN), a local area network (LAN), a wide area network (WAN), a metropolitan area network (MAN), a telephone network (e.g. the Public Switched Telephone Network (PSTN)), an ad hoc network, an intranet, the Internet, a fiber optic-based network, a satellite network, a cloud computing network, and/or a combination of these or other types of networks. Various data may be sent and received over network 120 between the various devices listed above, including user computing device(s) 121, caregiver computing device(s) 125, and physician computing device(s) 124.

Various other elements may be included in medication dispensing unit 102. Embodiments of the method and system described in the present disclosure include corresponding computer systems, devices, and computer programs recorded on one or more computer storage devices, each configured to perform particular operations or actions by virtue of having software, firmware, hardware, or a combination of them installed on the system and its respective components that an operator causes or cause the system to perform the actions. One or more computer programs can be configured to perform particular operations or actions by virtue of including instructions that may be executed by data processing units that are operable in the system.

Turning to FIG. 2, FIG. 2 is a pictorial illustration of an exemplary medication dispensing unit in accordance with one or more embodiments. Medication dispensing unit 202 may be an exemplary embodiment of medication dispensing unit 101 as shown in FIG. 1 and as described above. Only some elements of medication dispensing unit 202 are shown in FIG. 2, which is provided for illustrative example purposes only, and is not restricted to the appearance or form shown in FIG. 2. Thus, some or all of the components described above with respect to FIG. 1 and shown in the block diagram of FIG. 1 may also be present for medication dispensing unit 202.

According to one embodiment, medication dispensing unit 202 may be configured having a body and overall structure that is composed of a set of compartments in accordance with medication compartments 105 in FIG. 1. In this embodiment, each of medication compartments 105 in FIG. 1 may be in one of several levels of compartments. For example, outer level 204, intermediate level 206, and inner level 224 may all be a series or set of levels including individualized compartments (e.g. 105) for containing or holding medications (e.g. pills, tablets, gel capsules).

In one or more embodiments, medication dispensing unit 202 may be configured to have three levels (e.g. 204, 206, and 224). Alternatively, medication dispensing unit 202 may be configured to have two levels. Further, in additional embodiments, medication dispensing unit 202 may be configured so that additional levels may be connected as needed to the existing levels of medication dispensing unit 202. Accordingly, if a patient has a greater number of medications that need to be stored in medication dispensing unit 202, it may be possible to add additional levels. Further, each level may be shaped in a generally circular or disc like shape (e.g. as shown in FIG. 2). Thus, the set of medication compartments 105 of FIG. 1 may be configured to be a series of discs having individualized storage containers. Alternatively, the set of medication compartments 105 of FIG. 1 may be configured to be a series of rows and columns having individualized storage containers.

With respect to outer level 204, outer level 204 in FIG. 2 is depicted as including six medication compartments, such as medication compartment 226 which may represent each compartment in the outer level 204. Intermediate level 206 is depicted in FIG. 2 as also having six medication compartments, such as medication compartment 210 which may also represent each compartment in the intermediate level 206. Nevertheless, those of ordinary skill in the art will appreciate that more or less medication compartments may be included in alternative embodiments, and that outer level 204 and intermediate level 206 are not limited to this amount of individualized medication compartments (e.g. 226 and 210).

In one embodiment, a patient (or other provider or user) may place the different tablets or pills into each compartment of either outer layer 204 or intermediate layer 206 of medication dispensing unit 202. A patient (or caregiver) may pre-sort the medication when initially loading each compartment of outer layer 204 and intermediate layer 206 based on the kind or type of medication and also based on the timing of the medication with respect to the patient's prescribed medication schedule. Thus, a patient may place and store a dose of medication in each of medication compartments 226 in outer level 204 that is meant to be administered at a first time, and then may place a dose of medication in each of medication compartments 210 in intermediate level 206 that has a second time for administration and consumption which is different from the first. For example, the medication located in one of medication compartments 226 may be preferably consumed at 8:00 am, and the medication located in one of medication compartments 210 may be preferably consumed with food or water every four hours. Medication dispensing unit 202 is configured to include a processing unit (such as processor 109 of FIG. 1) and other electronic components that are able to dispense the medication from medication compartments 226 and 210 at these preferred times.

Accordingly, each medication compartment (e.g. 210 and 226) may be in electrical communication with a processing unit (and other components) configured to coordinate the release of the medication located in medication compartments with the preferred medication schedule for the patient. Further, medication dispensing unit 202 is able to dispense according to complex medication schedules. For example, once every two hours, once every four hours, once every eight hours, once every twelve hours, or any other interval as prescribed by a physician. Alternatively, the timing for the medication may be twice daily (e.g. AM and PM). Notably, the patient is able to store as many different types of medications as needed and to configure medication dispensing unit 202 to dispense the medication according to the prescribed schedule. Conventional medication dispensing units are not able to provide such versatility in dispensing medications in such amounts and according to complex timing and dispensation schedules.

Inner level 224 may also be included with medication dispensing unit 202. Inner level 224 may include additional medication compartments, such as medication compartments 212, 214, 216, and 218. Those of ordinary skill in the art will appreciate that a greater or lesser number of medication compartments may be used in alternative embodiments. Medication compartments 212, 214, 216, and 218 may be used to store medications in one embodiment.

Alternatively, in another embodiment, medication compartments 212, 214, 216, and 218 may be empty compartments and the medication located in the upper level compartments (e.g. 226 from outer level 204 and 210 from intermediate level 206) may be configured to be moved downwardly from these upper levels to one of the medication compartments of the inner level (e.g. 212, 214, 216, and 218) and then dispensed directly to the patient from any one of these medication compartments.

Accordingly, in one embodiment, outer level 204 and intermediate level 206 may be configured to rotate (either automatically or manually). As shown by the arrows in FIG. 2, outer level 204 may be configured to rotate in a clockwise direction and intermediate level 206 may be configured to rotate in a counterclockwise direction in one non-limiting embodiment. Alternatively, both outer level 206 and inner level 204 may be configured to rotate in the same direction or both directions (either clockwise or counterclockwise).

Further, in one or more embodiments, in response to a trigger or signal from a processor (e.g. processor 109) of medication dispensing unit 202, medication located in medication compartment 226 of outer level 204 may be moved to the medication compartment 210 of inner level 206 and then into medication compartment 212 in one non-limiting example. This movement of the medication to descend from a higher level to a lower level may be accomplished through various means and methods. In some embodiments, by rotating the outer level 204 and/or the intermediate level 206 to align with a medication compartment that has an interior opening through which the medications can slide into, the medications may descend to the lower level dispensing compartments due to gravity causing this downward movement toward the dispensing compartments of inner level 224.

In one embodiment, the lids of each compartment of outer level 204 and intermediate level 206 may lock (after being initially open and after the patient has placed the medication in the initial setting up of the medication dispensing unit 202). Upon dispensing the medication at a particular desired time from one of these upper level compartments, only a particular compartment (e.g. 212) from inner level 224 may be opened by a patient. This feature may be included so that the patient is not provided with access to all of the medications at any one time located in medication dispensing unit 202.

Further, as noted above, a fingerprint scanner (e.g. fingerprint scanner 116 in FIG. 1) may be included with medication dispensing unit 202 so that the user has to first provide a user's finger 220 on the fingerprint scanner before being able to perform various actions with medication dispensing unit 202 and before being granted access to medications dispensed from any of the medication compartments of outer level 204, intermediate level 206, and/or inner level 224. In this manner, the patient is restricted from being able to access any and all medications unless the patient is following the prescribed and recommended medical regimen provided by the physician. Further, other users are not able to gain access to the medications without authorization, which should reduce medication abuse.

Medication dispensing unit 202 may include a user interface (not shown in FIG. 2) and one or more displays (not shown) for a user to enter his or her measurements for the patient's pulse, oxygen saturation level, and/or blood pressure. In some embodiments, the patient may be able to enter this information directly into medication dispensing unit 202, whereby a processor (or other component) of medication dispensing unit 202 determines whether the vital sign measurements provided by the patient are acceptable for a particular medication to be released to the patient or not.

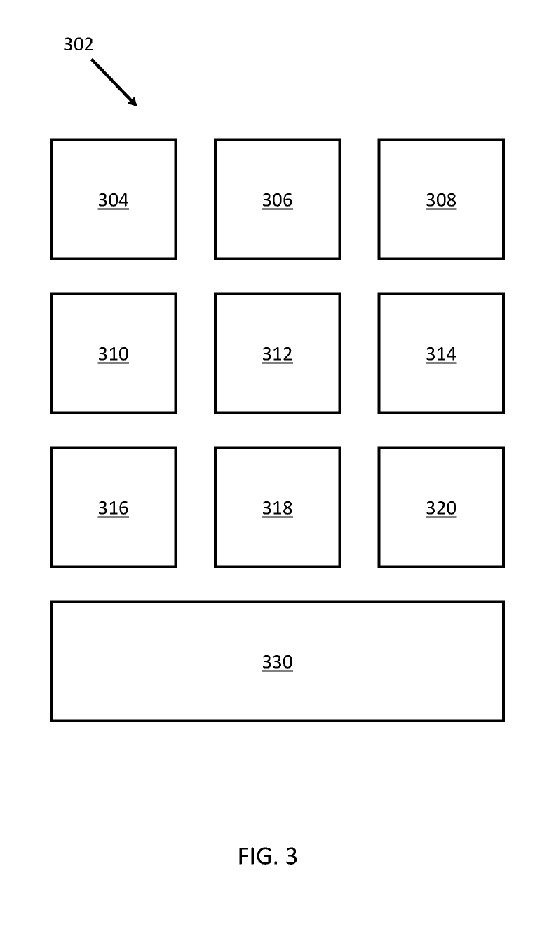

Turning to FIG. 3, FIG. 3 is another pictorial illustration of an exemplary medication dispensing unit in accordance with one or more embodiments. Medication dispensing unit 302 may be an exemplary embodiment of medication dispensing unit 101 as shown in FIG. 1 and as described above. Only some elements of medication dispensing unit 302 are shown in FIG. 3, which is provided for illustrative example purposes only, and is not restricted to the appearance or form shown in FIG. 3. Thus, some or all of the components described above with respect to FIG. 1 and shown in the block diagram of FIG. 1 may also be present for medication dispensing unit 302.

According to one or more embodiments, doses of medication for a patient are not pre-sorted into separate compartments. Instead, each of medication compartments 105 in FIG. 1 may contain one kind of medicine. For example, if a certain dose of medication is composed of sub-doses of pills of kind A, B, C, and D, compartment 304 may contain enough number of A pills to accommodate a variety of medication regimens for desired duration of medication. Likewise, compartment 306 may contain enough number of B pills to accommodate a variety of medication regimens for desired duration of medication, and so forth. The number of compartments 304-320 shown in FIG. 3 is only illustrative, and there may be as many compartments as needed by specific medication and specific medication schedule for a patient. As in other embodiments described above, the lids of compartments 304-320 may lock (after being initially open and after the patient has placed the medication in the initial setting up of the medication dispensing unit 302).

In these embodiments, at a specific dispensing time, a processing unit (not shown) such as processor 109 of FIG. 1 may be configured to open the lid of one of the compartments 304-320, extract a pre-determined sub-dose of medicine for the corresponding dispensing time, dispense the sub-dose of medicine to another medication compartment, for example to dispensing tray 330.

After the processing unit dispenses all the sub-doses of medicines that make up the dose of medication of the corresponding dispensing time, the dose of medication is ready for the patient to take. Dispensing tray 330 may also be locked until a proper identification of the patient is confirmed by, for example, a fingerprint scanning, as described above. In some embodiments, medication dispensing unit 302 may have more than one dispensing trays 330 such that the lids of dispensing trays 330 may be open at different time of the day. For example, there may be three dispensing trays 330, each for morning, afternoon, and evening medication, respectively. Also, the patient may be notified when a certain dose of medication is ready in a specific dispensing tray 330 in various ways as described above for embodiments in FIG. 1.

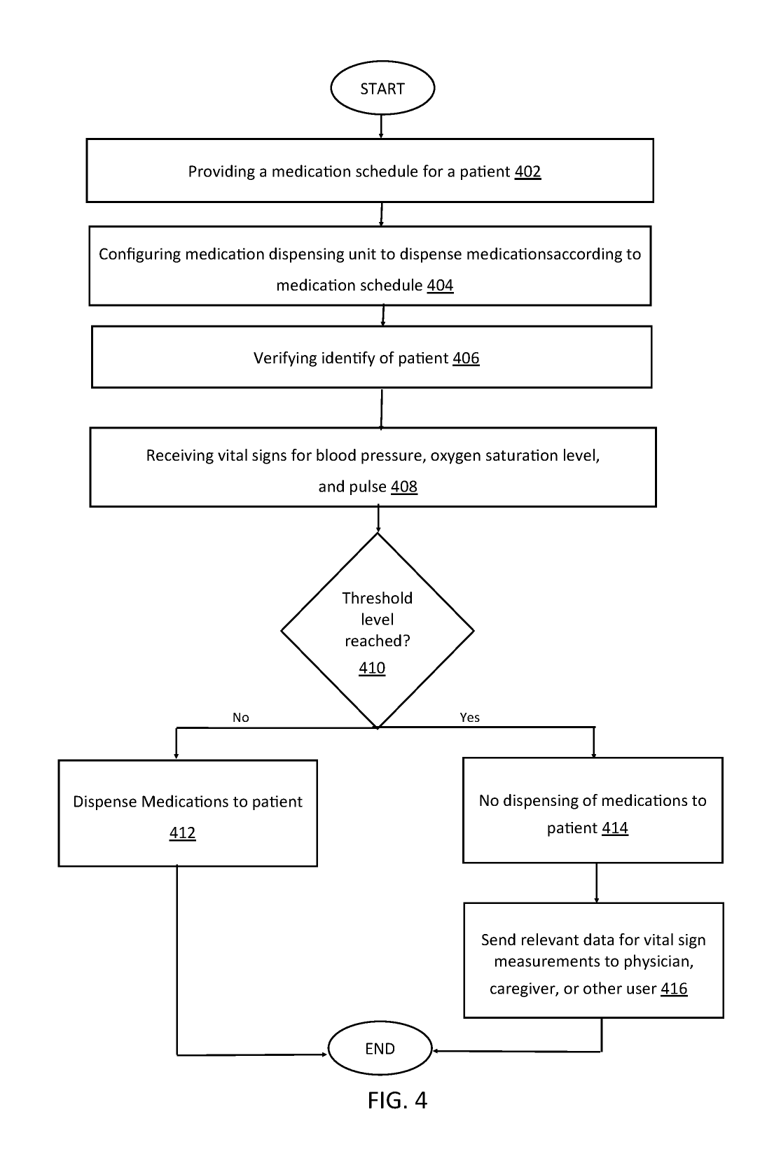

Turning to FIG. 4, FIG. 4. is a flowchart of an exemplary process for storing and dispensing medication from a medication dispensing unit. The medication dispensing unit may be in accordance with medication dispensing units 102 in FIG. 1, and medication dispensing unit 202 in FIG. 2 or medication dispensing unit 302 in FIG. 3.

At step 402, the process may include providing a medication schedule for a patient. The medication schedule may be provided by a physician or group of physicians. Further, the medications may include recommendations for nutritional supplements, including vitamins or other types of supplements. At step 404, the process may include configuring the medication dispensing unit to dispense the medication for the patient according to the medication schedule of the patient. As discussed above, a medication dispensing unit, such as medication dispensing units 102, 202 and 302, may include one or more processors that are useful for executing instructions for storing and dispensing the medications according to a prescribed schedule. In some embodiments, this process may include arranging and distributing the medication in one or more medication compartments located on one or more levels of the medication dispensing unit. In some embodiments, this process may include setting the timing and dosage amount of any medication to be dispensed from a particular medication compartment according to the system used for dispensing such medication.

Further, the medication dispensing units, such as medication dispensing units 102 and 202, may include one or more mechanisms for manipulating a position of a medication from an outer or intermediate level (e.g. 204 and 206) to compartments of an inner level (e.g. inner level 224 and its respective compartments 212-218). As described above, this may include causing the medication stored in upper level compartments to descend due to gravity or some other mechanism to lower level dispensing compartments. Accordingly, in such embodiments, medication dispensing units may be configured to have a series or set of connectable levels, such as set of connectable levels between medication compartments 105 in medication dispensing unit 102. Further, the medication dispensing unit may be configured to have a set of connecting disks that include a set amount of medication per disk.

In some embodiments, the medication dispensing units, such as medication dispensing units 102 and 302, may include one or more mechanisms for manipulating position of medicines from one compartment to another, such as dispensing tray 330. As described above, this may include a mechanism for extracting a pre-determined sub-dose of medicine from each medication compartment for the corresponding dispensing time.

At step 406, the process may include verifying a user's (i.e. patient's) proper identity. As discussed above, a medication dispensing unit may include one or more biometric devices, such as biometric sensors 115, which includes fingerprint scanner 116, such that a user must first scan his or her fingerprint to be provided access to the medications of the medication dispensing unit.

To continue with an exemplary process, at step 408, the process may include receiving measurements regarding a patient's blood pressure level, oxygen saturation level, and pulse. In some embodiments, the medication dispensing units may be configured to connect to one or more medical devices such as blood pressure monitoring device 118 and pulse oximeter 119. Further, the readings may be supplied directly and automatically blood pressure monitoring device 118 and pulse oximeter 119. Alternatively, or additionally, the patient or another user or person may enter the biological characteristics including vital signs using a user interface, such as user interface 107, and a display, such as display 108 located on the medication dispensing unit.

At step 410, the process may verify whether the vital sign measurements meet a threshold level that is concerning or potentially dangerous for receiving medications from the medication dispensing unit. If the answer is yes, then the medication dispensing unit may be configured to not dispense some or all of the stored medications (at step 414). Further, the process may continue by notifying a patient's physician, caregiver, or other health care provider of the concerning measurement for the oxygen saturation level, pulse, and/or blood pressure (step 416).

However, if the answer to the query is no (whereby the vital signs are normal and/or not concerning because they do not meet the threshold trigger point), then the medications are dispensed from the medication dispensing unit to the patient according to his or her normal schedule (step 412). Additional elements may be required for a medication dispensing unit to dispense medications. The process shown and described for FIG. 4 is exemplary and non-restrictive of additional steps being included or some steps being eliminated, such as repeating steps 406 through 416 as prescribed throughout the day or week until a prescription has changed.

Accordingly, the present description includes one or more embodiments for a medication dispensing unit that includes advantages over conventional methods for storing and dispensing medications to a user. In one or more embodiments, a “smart” medication dispensing unit is provided that can handle a patient's complex medication schedule and for an unlimited and/or high number of medications (e.g. 7 or 8 medications or more). Further, the embodiments described herein may include a medication dispensing unit that prevents access to the medications if a patient does not first verify his or her identify (e.g. using a fingerprint scanning device), which helps prevent medication abuse and misuse. Additionally, the one or more embodiments provided herein may first require a patient to provide or for the information from a set of medication devices (e.g. a pulse oximeter and/or blood pressure monitoring device) to be provided to the medication dispensing unit in order to prevent any adverse health effects if the patient does take the medication while his or her state of health is not optimal, as indicated by the concerning vital sign levels. Those of ordinary skill in the art will appreciate that there may be many applications and uses for such medication dispensing units as described above.

While the present invention has been related in terms of the foregoing embodiments, those skilled in the art will recognize that the invention is not limited to the embodiments described. The present invention may be practiced with modification and alteration within the spirit and scope of the appended claims. Thus, the description is to be regarded as illustrative instead of restrictive on the present invention.

While preferred and alternate embodiments have been illustrated and described, as noted above, many changes can be made without departing from the spirit and scope of the embodiments provided in the present disclosure. Accordingly, the scope of the embodiments provided in the present disclosure is not limited by the disclosure of these preferred and alternate embodiments. Instead, the scope of the invention title be determined entirely by reference to the claims. Insofar as the description above and the accompanying drawings (if any) disclose any additional subject matter that is not within the scope of the claims below, the inventions are not dedicated to the public and Applicant hereby reserves the right to file one or more applications to claim such additional inventions.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example of a generic series of equivalent or similar features.

Any element in a claim that does not explicitly state “means for” performing a specified function, or “step for” performing a specific function is not to be interpreted as a “means” or “step” clause as specified in 35. U.S.C. § 112 ¶ 6. In particular, the use of “step of” in the claims herein is not intended to invoke the provisions of U.S.C. § 112 ¶ 6.

What is claimed is:

1. A system for dispensing medication, the system comprising:
a plurality of compartments to contain medication for a patient, wherein the compartments are configured to dispense one or more pre-determined doses of medication of the patient;
one or more biometric sensors configured to measure one or more of biological characteristics of the patient;
a display configured to display medication-related information;
one or more processors configured to:
determine one or more dispense times, each dispense time corresponding to each dose of the pre-determined doses of medication;
store one or more parameters for dispensing the pre-determined doses of medication at the corresponding dispense times, wherein the parameters relate to the biological characteristics;
receive the biological characteristics of the patient from the one or more biometric sensors;
compare the parameters with the biological characteristics received;
determine whether to dispense one or more of the pre-determined doses of medication from the compartments; and
dispense one or more of the pre-determined doses of medication from the compartments.

2. The system of claim 1, wherein the one or more biometric sensors comprise a fingerprint scanning device.

3. The system of claim 1, wherein the one or more biometric sensors comprise a pulse oximeter and a blood pressure monitoring device.

4. The system of claim 3, wherein the biological characteristics comprise a blood pressure, an oxygen saturation level, and a pulse measured from the pulse oximeter and the blood pressure monitoring device.

5. The system of claim 4, wherein the one or more processors are further configured to:
determine whether any one of the received or entered blood pressure measurement, oxygen saturation level, or pulse is at a threshold level; and
prevent dispensing of the pre-determined doses of medication from the compartments, if a determination is made that the blood pressure measurement, oxygen saturation level, or pulse measurement is at a threshold level.

6. The system of claim 1, further comprising a user interface configured to receive one or more user inputs comprising a patient identification.

7. The system of claim 6, wherein the one or more user inputs further comprise a patient medical record.

8. The system of claim 1, wherein the medication-related information comprises a reminder to the patient of each of the dispense times.

9. The system of claim 8, wherein the one or more processors are further configured to send the reminder to the patient, wherein the reminder is at least one of a text message, audible alarm, visual alarm, or email message.

10. The system of claim 1, wherein the medication-related information comprises instructions for taking the medication.

11. The system of claim 1, wherein the medication-related information comprises informational literature about the medication for the patient.

12. The system of claim 1, further comprising a computing device, wherein the computing device is in electronic communication with the one or more processors over a network.

13. The system of claim 12, wherein the one or more processors are configured to send medication-related information to the computing device.

14. The system of claim 1, wherein each of the plurality of compartments contains a dose of the pre-determined doses of medication.

15. The system of claim 1, wherein the one or more processors are further configured to:
open one of the plurality of compartments; and
extract the dose contained in the one of the plurality of compartments.

16. The system of claim 1, wherein
each dose of the pre-determined doses of medication comprises a combination of different kinds of medicine, each of the different kinds of medicine having a pre-determined sub-dose, and
each of the plurality of compartments contains one of the different kinds of medicine.

17. The system of claim 16, wherein the one or more processors are further configured to:
open a first compartment of the plurality of compartments;
extract a first pre-determined sub-dose of the medicine from the first compartment;
open a second compartment of the plurality of compartments;
extract a second pre-determined sub-dose of the medicine from the second compartment; and
combine the first second pre-determined sub-dose with the second pre-determined sub-dose.

18. A method for storing and dispensing medication from a medication dispensing unit, comprising:
providing one or more dispense times for a patient;
configuring the medication dispensing unit to dispense the medication to the patient according to the dispense times for the patient;
verifying an identify of the patient; and
configuring the medication dispensing unit to receive one or more biological characteristics of the patient prior to dispensing particular medication for the patient, further comprising:
receiving biological characteristics comprising blood pressure level, oxygen saturation level, and pulse of the patient;
determining whether a threshold trigger level is reached for at least one of the received blood pressure level, oxygen saturation level, and pulse of the patient;
if the threshold trigger level is reached, then not dispensing the medication to the patient and providing the received biological characteristics to a healthcare provider of the patient;
if the threshold trigger level is not reached, then dispensing the medication to the patient according to the dispense times.

19. The method of claim 18, wherein receiving the biological characteristics further comprises connecting the medication dispensing unit to a blood pressure monitoring device and a pulse oximeter.

20. The method of claim 18, wherein verifying the identity of the patient further comprises:
associating a fingerprint of the patient with the medication dispensing unit; and
verifying the fingerprint of the patient prior to dispensing the medication from the medication dispensing unit.
